SEQUENCE LISTING (2 sequences)

SEQ ID NO: 1 - DNA, 2010 bp, Homo sapiens
gtgctgccgcgggtcacgtgctgcggaggcttggggaggggcggcgaggcggggtttatagcccgggcgcccgcgggccccacgctttgaccgggtcgtggcagccggagtcgtcttcgggacgcgcctgctcttcgcctttcgctgcagtccgtcgatttctttctccaggaagaaaaatggcatccgttgcagttgatccacaaccgagtgtggtgactcgggtggtcaacctgcccttggtgagctccacgtatgacctcatgtcctcagcctatctcagtacaaaggaccagtatccctacctgaagtctgtgtgtgagatggcagagaacggtgtgaagaccatcacctccgtggccatgaccagtgctctgcccatcatccagaagctagagccgcaaattgcagttgccaatacctatgcctgtaaggggctagacaggattgaggagagactgcctattctgaatcagccatcaactcagattgttgccaatgccaaaggcgctgtgactggggcaaaagatgctgtgacgactactgtgactggggccaaggattctgtggccagcacgatcacaggggtgatggacaagaccaaaggggcagtgactggcagtgtggagaagaccaagtctgtggtcagtggcagcattaacacagtcttggggagtcggatgatgcagctcgtgagcagtggcgtagaaaatgcactcaccaaatcagagctgttggtagaacagtacctccctctcactgaggaagaactagaaaaagaagcaaaaaaagttgaaggatttgatctggttcagaagccaagttattatgttagactgggatccctgtctaccaagcttcactcccgtgcctaccagcaggctctcagcagggttaaagaagctaagcaaaaaagccaacagaccatttctcagctccattctactgttcacctgattgaatttgccaggaagaatgtgtatagtgccaatcagaaaattcaggatgctcaggataagctctacctctcatgggtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgtaccacagaacatccaagatcaagccaagcacatgggggtgatggcaggcgacatctactcagtgttccgcaatgctgcctcctttaaagaagtgtctgacagcctcctcacttctagcaaggggcagctgcagaaaatgaaggaatctttagatgacgtgatggattatcttgttaacaacacgcccctcaactggctggtaggtcccttttatcctcagctgactgagtctcagaatgctcaggaccaaggtgcagagatggacaagagcagccaggagacccagcgatctgagcataaaactcattaaacctgcccctatcactagtgcatgctgtggccagacagatgacaccttttgttatgttgaaattaacttgctaggcaaccctaaattgggaagcaagtagctagtataaaggccctcaattgtagttgtttccagctgaattaagagctttaaagtttctggcattagcagatgatttctgttcacctggtaagaaaagaatgataggcttgtcagagcctatagccagaactcagaaaaaattcaaatgcacttatgttctcattctatggccattgtgttgcctctgttactgtttgtattgaataaaaacatcttcatgtgggctggggtagaaactggtgtctgctctggtgtgatctgaaaaggcgtcttcactgctttatctcatgatgcttgcttgtaaaacttgattttagtttttcatttctcaaataggaatactacctttgaattcaataaaattcactgcaggatagaccagttaaaaaaaaaaaaaaaaaaaaaaaaaaaaaaa

SEQ ID NO: 2 - protein, 437 aa, Homo sapiens
MASVAVDPQPSVVTRVVNLPLVSSTYDLMSSAYLSTKDQYPYLKSVCEMAENGVKTITSVAMTSALPIIQKLEPQIAVANTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEHIESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

(19) United States
(12) Patent Application Publication
(10) Pub. No.: US 2011/0213009 A1
(43) Pub. Date: Sep. 1, 2011

(54) ADFP MODULATORS IN THE TREATMENT OF ACNE, OF SEBORRHOEIC DERMATITIS OR OF HYPERSEBORRHOEA

(75) Inventors: Jérôme Aubert, Grasse (FR); Sophie Deret, Mougins (FR); Valérie Trinquet, Antibes (FR)

(73) Assignee: Galderma Research & Development, Biot (FR)

(21) Appl. No.: 12/991,329

(22) PCT Filed: May 7, 2009
(86) PCT No.: PCT/EP09/55552

§ 371 Date: Feb. 25, 2011

Related U.S. Application Data
(60) Provisional application No. 61/071,595, filed on May 7, 2008.

(30) Foreign Application Priority Data
Nov. 13, 2008 (FR) 0857694

(51) Int. Cl. A61K 31/713; C12Q 1/68; C07H 21/04; C40B 30/04; G01N 33/53; C07H 21/02; A61K 31/56; A61P 17/00; A61P 17/10; A61P 17/08

(52) U.S. Cl. 514/44 A; 435/6.13; 536/23.5; 506/9; 435/7.92; 536/24.5; 514/171

(57) ABSTRACT

An in vitro or in vivo method for screening for candidate compounds for the preventive or curative treatment of acne, of seborrhoeic dermatitis or of skin disorders associated with hyperseborrhoea, includes determining the ability of a compound to modulate the expression or the activity of the adipose differentiation-related protein (ADFP), and also utilizes modulators of the expression or of the activity of this protein, for the treatment of acne, of seborrhoeic dermatitis or of skin disorders associated with hyperseborrhoea; methods for the in vitro diagnosis of or in vitro prognosis for these pathologies are also featured.

The invention relates to the identification and the use of compounds which modulate the adipose differentiation-related protein (ADFP) for treating acne, seborrhoeic dermatitis, and also skin disorders associated with hyperseborrhoea. It also relates to methods for the in vitro diagnosis of or in vitro prognosis for these pathologies.

Hyperseborrhoeic greasy skin is characterized by exaggerated secretion and excretion of sebum. Conventionally, a sebum level greater than 200 μg/cm² measured on the forehead is considered to be characteristic of greasy skin. Greasy skin is often associated with a desquamation deficiency, a glistening complexion and a thick skin grain. In addition to these aesthetic disorders, excess sebum can serve as a support for the anarchical development of saprophytic bacterial flora (P. acnes in particular), and cause the appearance of comedones and/or acneic lesions.

This stimulation of sebaceous gland production is induced by androgens.

Acne is, in fact, a chronic disease of the pilosebaceous follicle under hormonal control. Hormone therapy against acne is one treatment possibility for women, the objective being to prevent the effects of androgens on the sebaceous gland. In this context, oestrogens, anti-androgens or agents which reduce the production of androgens by the ovaries or the adrenal gland are generally used. The anti-androgens used for the treatment of acne include, in particular, spironolactone, cyproterone acetate and flutamide. However, these agents have potentially severe side effects. Thus, any pregnancy must be absolutely prevented, in particular because of a risk of feminization for the male foetus. These agents are prohibited in male patients.

Seborrhoeic dermatitis is a common inflammatory skin dermatosis which presents in the form of red plaques covered with greasy, yellowish squames, which are more or less pruriginous, and are predominant in the seborrhoeic areas.

A need therefore exists, for these diseases, to identify mediators downstream of the action of the steroid hormones, and to modulate them, in order to obtain a similar therapeutic profile, but with reduced side effects.

The Applicant has now discovered that the gene encoding the adipose differentiation-related protein (ADFP) is expressed preferentially in human sebaceous glands in comparison with the epidermis, and that the expression thereof is regulated in vitro by a cocktail which promotes the differentiation of sebocyte precursors, containing an androgen (R1881, also known as methyltrienolone, at 1 nM) and a PPARγ ligand (Rosiglitazone, which is 6-(2-methoxyethoxy-methoxy)naphthalene-2-carboxylic acid [4′-(2,4-dioxothiazolidin-5-ylmethyl)biphenyl-3-ylmethyl]methyl-amide, at 100 nM), in a primary culture of human sebocytes.

It consequently proposes targeting the ADFP gene or the expression product thereof, for preventing and/or improving acne, seborrhoeic dermatitis or skin disorders associated with hyperseborrhoea, in particular the greasy skin appearance.

It is, moreover, known that treatment with a PPAR agonist induces a large decrease in the size of the sebaceous glands, and a reduction in androgen-induced hyperseborrhoea (WO2007/093747).

Since the target proposed is downstream of the PPAR receptor, it is said target which is responsible for the effects observed on the sebaceous glands and on sebum excretion.

Thus, the gene identified can be used to identify the compounds which are the most active as PPAR modulators, to classify them and to select them. On this basis, it is also proposed to use the ADFP gene or the ADFP protein as a marker for screening for candidate PPAR modulators for the treatment of acne, seborrhoeic dermatitis or a skin disorder associated with hyperseborrhoea. More specifically, the ability of a PPAR modulator to modulate the expression or the activity of ADFP or the expression of the gene thereof or the activity of at least one of the promoters thereof, can be determined.

The term “acne” is intended to mean all the forms of acne, i.e. in particular acne vulgaris, comedonal acne, polymorphous acne, nodulocystic acne, acne conglobata, or else secondary acne such as solar acne, acne medicamentosa or occupational acne. The Applicant also proposes methods of in vitro, in vivo and clinical diagnosis or prognosis based on the detection of the level of expression or of activity of ADFP.

### ADFP

The term “ADFP” denotes the adipose differentiation-related protein, also known as ADRP, Adipophilin or MGC10598. The adipose differentiation-related protein is a major constituent of the surface of lipid globules. ADFP is involved in the storage of lipids in the form of lipid droplets. An increase in the level of its messenger RNA is one of the early indications of adipocyte differentiation (Jiang et al. 1992, PNAS, 89(17):7856-60).

ADFP expression is also induced in vivo in models of skin irritation, and also in vitro in keratinocytes treated with irritant agents (Corsini et al, 2002, Toxicol In Vitro, 16(4):427-31; Corsini et al, 2003, J Invest Dermatol, 121(2):337-44). The inhibition of ADFP expression through an antisense oligonucleotides approach is accompanied by a decrease in hepatosteatosis (Imai et al, 2007, Gastroenterology, 132(5):1947-54) and by an induction of cell cytotoxicity.

In the context of the invention, the term “ADFP gene” or “ADFP nucleic acid” signifies the gene or the nucleic acid sequence which encodes the adipose differentiation-related protein. While the target aimed for is preferably the human gene or the expression product thereof, the invention may also call upon cells expressing a heterologous adipose differentiation-related protein, by genomic integration or transient expression of an exogenous nucleic acid encoding the protein.

The human cDNA sequence of ADFP is reproduced in the annex (SEQ ID No. 1). It is the sequence NM_{—}001122 (Genbank), the open reading frame of which contains 2010 base pairs and encodes a 48.1 KDa protein.

### Diagnostic Applications

A subject of the invention concerns an in vitro method for diagnosing or monitoring the development of acneic lesions, seborrhoeic dermatitis or a skin disorder associated with hyperseborrhoea in an individual, comprising the comparison of the expression or of the activity of the adipose differentiation-related protein (ADFP), of the expression of the gene thereof or of the activity of at least one promoter thereof, in a biological sample from an individual, with respect to a biological sample from a control individual.

The protein expression can be determined by assaying the ADFP protein according to one of the methods such as Western blotting, immunohistochemistry, mass spectrometry analysis (Maldi-TOF and LC/MS analysis), radioimmunoassay (RIA) and ELISA or any other method known to those skilled in the art. Another method, in particular for measuring the expression of the ADFP gene, is to measure the amount of corresponding mRNA. Assaying of the ADFP activity can also be envisaged.

In the context of a diagnosis, the “control” individual is a “healthy” individual.

In the context of monitoring the development of acneic lesions, of seborrhoeic dermatitis or of a skin disorder associated with hyperseborrhoea, the “control individual” refers to the same individual at a different time, which preferably corresponds to the beginning of the treatment (T0). This measurement of the difference in expression or in activity of ADFP, or in expression of the gene thereof or in activity of at least one promoter thereof, makes it possible in particular to monitor the effectiveness of a treatment, in particular a treatment with an ADFP modulator, as envisaged above, or another treatment against acne, seborrhoeic dermatitis or a skin disorder associated with hyperseborrhoea. Such monitoring can reassure the patient with regard to whether continuing the treatment is well-founded or necessary.

Another aspect of the present invention concerns an in vitro method for determining an individual's susceptibility to developing acneic lesions, seborrhoeic dermatitis or a skin disorder associated with hyperseborrhoea, comprising the comparison of the expression or of the activity of the ADFP protein, of the expression of the gene thereof or of the activity of at least one of the promoters thereof, in a biological sample from an individual, with respect to a biological sample from a control individual.

Here again, the expression of the ADFP protein can be determined by assaying this protein by immunoassay, for example by ELISA assay, or by any other method mentioned above. Another method, in particular for measuring the expression of the ADFP gene, is to measure the amount of corresponding mRNA by any method as described above. Assaying of the ADFP activity can also be envisaged.

The individual tested is in this case an asymptomatic individual exhibiting no skin condition associated with hyperseborrhoea, seborrhoeic dermatitis or acne. The “control” individual in this method signifies a “healthy” reference population or individual. The detection of this susceptibility makes it possible to set up a preventive treatment and/or increased monitoring of the signs associated with acne, seborrhoeic dermatitis or a skin disorder associated with hyperseborrhoea.

In these in vitro diagnostic or prognostic methods, the biological sample tested may be any sample of biological fluid or a sample of a biopsy. Preferably, the sample may be a preparation of skin cells, obtained for example by desquamation or biopsy. It may also be sebum.

### Screening Methods

A subject of the invention is an in vitro or in vivo method for screening for candidate compounds for the preventive and/or curative treatment of acne, of seborrhoeic dermatitis or of any skin disorder associated with hyperseborrhoea, comprising the determination of the ability of a compound to modulate the expression or the activity of the adipose differentiation-related protein or the expression of the gene thereof or the activity of at least one of the promoters thereof, said modulation indicating the usefulness of the compound for the preventive or curative treatment of acne, seborrhoeic dermatitis or any skin disorder associated with hyperseborrhoea. The method therefore makes it possible to select the compounds capable of modulating the expression or the activity of ADFP, or the expression of the gene thereof, or the activity of at least one of the promoters thereof.

More particularly, the subject of the invention is an in vitro method for screening for candidate compounds for the preventive and/or curative treatment of acne, of seborrhoeic dermatitis or of skin disorders associated with hyperseborrhoea, comprising the following steps:
a. preparing at least two biological samples or reaction mixtures;
b. bringing one of the samples or reaction mixtures into contact with one or more of the test compounds;
c. measuring the expression or the activity of the adipose differentiation-related protein, the expression of the gene thereof or the activity of at least one of the promoters thereof, in the biological samples or reaction mixtures;
d. selecting the compounds for which a modulation of the expression or of the activity of the adipose differentiation-related protein, of the expression of the gene thereof or of the activity of at least one of the promoters thereof, is measured in the sample or the mixture treated in b), compared with the untreated sample or with the untreated mixture.

An in vivo screening method can be carried out in any laboratory animal, for example, a rodent. According to one preferred embodiment, the screening method comprises administering the test compound to the animal preferably by topical application, then optionally sacrificing the animal by euthanasia, and taking a sample of an epidermal split, before evaluating the expression of the gene in the epidermal split, by any method described herein.

The term “modulation” is intended to mean any effect on the expression or the activity of the protein, the expression of the gene or the activity of at least one of the promoters thereof, i.e. optionally a stimulation, but preferably a partial or complete inhibition. Thus, the compounds tested in step d) above preferably inhibit the expression or the activity of the adipose differentiation-related protein, the expression of the gene thereof or the activity of at least one of the promoters thereof. The difference in expression obtained with the compound tested, compared with a control carried out in the absence of the compound, is significant starting from 25% or more.

Throughout the present text, unless otherwise specified, the term “expression of a gene” is intended to mean the amount of mRNA expressed;

the term “expression of a protein” is intended to mean the amount of this protein;

the term “activity of the ADFP protein” is intended to mean the ability of the protein to induce an accumulation of lipids in the form of lipid droplets and/or to adjust the efflux of lipids in the cytosol;

the term “activity of a promoter” is intended to mean the ability of this promoter to initiate the transcription of the DNA sequence encoded downstream of this promoter (and therefore indirectly the synthesis of the corresponding protein).

The compounds tested may be of any type. They may be of natural origin or may have been produced by chemical synthesis. This may involve a library of structurally defined chemical compounds, uncharacterized compounds or substances, or a mixture of compounds.

In particular, the invention is directed towards the use of the ADFP gene or of the ADFP protein, as a marker for candidate PPAR or AR (androgen receptor) modulators for treating acne, seborrhoeic dermatitis or a skin disorder associated with hyperseborrhoea. More specifically, the ability of a PPAR or AR modulator to modulate the expression or the activity of ADFP or the expression of the gene thereof or the activity of at least one of the promoters thereof is determined.

Preferably, the modulator is a PPARγ modulator.

The PPAR modulator is a PPAR agonist or antagonist, preferably an agonist.

The AR modulator is an AR agonist or antagonist, preferably an agonist.

Various techniques can be used to test these compounds and to identify the compounds of therapeutic interest which modulate the expression or the activity of the adipose differentiation-related protein.

According to a first embodiment, the biological samples are cells transfected with a reporter gene functionally linked to all or part of the promoter of the gene encoding the adipose differentiation-related protein, and step c) described above comprises measuring the expression of said reporter gene.

The reporter gene may in particular encode an enzyme which, in the presence of a given substrate, results in the formation of coloured products, such as CAT (chloramphenicol acetyltransferase), GAL (beta-galactosidase) or GUS (beta-glucuronidase). It may also be the luciferase gene or the GFP (green fluorescent protein) gene. The assaying of the protein encoded by the reporter gene, or of the activity thereof, is carried out conventionally by colorimetric, fluorometric or chemiluminescence techniques, inter alia.

According to a second embodiment, the biological samples are cells expressing the gene encoding the adipose differentiation-related protein, and step c) described above comprises measuring the expression of said gene.

The cell used herein may be of any type. It may be a cell expressing the ADFP gene endogenously, for instance an adipocyte, a keratinocyte, or better still a sebocyte. Organs of human or animal origin may also be used, for instance the preputial gland, the clitoral gland, or else the sebaceous gland of the skin.

It may also be a cell transformed with a heterologous nucleic acid encoding the preferably human, or mammalian, ADFP protein.

A large variety of host-cell systems may be used, such as, for example, Cos-7, CHO, BHK, 3T3 or HEK293 cells. The nucleic acid may be transfected stably or transiently, by any method known to those skilled in the art, for example by calcium phosphate, DEAE-dextran, liposome, virus, electroporation or microinjection.

In these methods, the expression of the ADFP gene or of the reporter gene can be determined by evaluating the level of transcription of said gene, or the level of translation thereof.

The expression “level of transcription of a gene” is intended to mean the amount of corresponding mRNA produced. The expression “level of translation of a gene” is intended to mean the amount of protein produced. Those skilled in the art are familiar with the techniques for quantitatively or semi-quantitatively detecting the mRNA of a gene of interest. Techniques based on hybridization of the mRNA with specific nucleotide probes are the most common (Northern blotting, RT-PCR (reverse transcriptase polymerase chain reaction), quantitative RT-PCR (qRT-PCR), RNase protection). It may be advantageous to use detection labels, such as fluorescent, radioactive or enzymatic agents or other ligands (for example, avidin/biotin).

In particular, the expression of the gene can be measured by real-time PCR or by RNase protection. The term “RNase protection” is intended to mean the detection of a known mRNA among the poly(A)-RNAs of a tissue, which can be carried out using specific hybridization with a labelled probe. The probe is a labelled (radioactive) RNA complementary to the messenger to be sought. It can be constructed from a known mRNA, the cDNA of which, after RT-PCR, has been cloned into a phage. Poly(A)-RNA from the tissue in which the sequence is to be sought is incubated with this probe under slow hybridization conditions in a liquid medium. RNA:RNA hybrids form between the mRNA sought and the antisense probe. The hybridized medium is then incubated with a mixture of ribonucleases specific for single-stranded RNA, such that only the hybrids formed with the probe can withstand this digestion. The digestion product is then deproteinated and repurified, before being analysed by electrophoresis. The labelled hybrid RNAs are detected by autoradiography.

The level of translation of the gene is evaluated, for example, by immunological assaying of the product of said gene. The antibodies used for this purpose may be of polyclonal or monoclonal type. The production thereof involves conventional techniques. An anti-ADFP polyclonal antibody can, inter alia, be obtained by immunization of an animal, such as a rabbit or a mouse, with the whole protein. The antiserum is taken and then depleted according to methods known per se to those skilled in the art. A monoclonal antibody can, inter alia, be obtained by the conventional method of Köhler and Milstein (Nature (London), 256: 495-497 (1975)). Other methods for preparing monoclonal antibodies are also known. Monoclonal antibodies can, for example, be produced by expression of a nucleic acid cloned from a hybridoma. Antibodies can also be produced by the phage display technique, by introducing antibody cDNAs into vectors, which are typically filamentous phages which display V-gene libraries at the surface of the phage (for example, fUSE5 for E. coli).

The immunological assaying can be carried out in solid phase or in homogeneous phase; in one step or in two steps; in a sandwich method or in a competition method, by way of nonlimiting examples. According to one preferred embodiment, the capture antibody is immobilized on a solid phase. By way of nonlimiting examples of a solid phase, use may be made of microplates, in particular polystyrene microplates, or solid particles or beads, or paramagnetic beads.

ELISA assays, radioimmunoassays or any other detection technique can be used to reveal the presence of the antigen/antibody complexes formed.

The characterization of the antigen/antibody complexes, and more generally of the isolated or purified, but also recombinant, proteins (obtained in vitro and in vivo) can be carried out by mass spectrometry analysis. This identification is made possible by virtue of the analysis (determination of the mass) of the peptides generated by enzymatic hydrolysis of the proteins (in general, trypsin). In general, the proteins are isolated according to the methods known to those skilled in the art, prior to the enzymatic digestion. The analysis of the peptides (in hydrolysate form) is carried out by separating of the peptides by HPLC (nano-HPLC) based on their physicochemical properties (reverse phase). The determination of the mass of the peptides thus separated is carried out by ionization of the peptides and either by direct coupling with mass spectrometry (electrospray ESI mode), or after deposition and crystallization in the presence of a matrix known to those skilled in the art (analysis in MALDI mode). The proteins are subsequently identified through the use of appropriate software (for example, Mascot).

According to a third embodiment, the screening method comprises bringing a compound into contact with an ADFP protein and determining the ability of the compound to modulate the activity of ADFP, a difference in activity, compared to a control carried out in the absence of the compound, indicating the usefulness of the compound for the preventive or curative treatment of acne, of seborrhoeic dermatitis or of skin disorders associated with hyperseborrhoea.

Preferably, the ability of the compound to bind the ADFP protein is also evaluated.

The determination of the ability of the compound to modulate the activity of ADFP can be carried out in various ways, for example by analysing the accumulation of lipids in the form of lipid droplets.

An analysis of ADFP-induced accumulation of lipids in lipid droplets has been described in the literature (Greenspan et al, 1985, J Cell Biol, 100:965-973). Thus, the accumulation of lipids in lipid droplets can, for example, be evaluated by fluorescence microscopy and flow cytometry in the following way: epidermal cells cultured beforehand in an RPMI medium and possibly treated with an irritant agent (SDS or sodium dodecyl sulphate) are washed with PBS (phosphate buffer), then subjected to trypsin and fixed in 2% formaldehyde at ambient temperature. The cells are then rinsed with PBS, permeabilized, and stained with Nil red (9-diethylamino-5H-benzo[alpha]phenoxazin-5-one) for 10 minutes. The Nil red is dissolved in ethanol at 1 mg/ml, and then diluted in PBS to a final concentration of 10 μg/ml. After rinsing, the cells are resuspended in 0.5 ml of PBS and a cytospin onto glass slides is carried out with 50 μl. The cells are marked for 30 minutes with an anti-human ADFP antibody coupled to FITC, diluted to 1/10. After rinsing, the cells are resuspended in 0.5 ml of PBS and a cytospin onto a glass slide is carried out with 50 μl. In both cases, the slides are observed under a fluorescence microscope.

The compounds selected by means of the screening methods defined herein can subsequently be tested on other in vitro models and/or in vivo models (in animals or humans) for their effects on acne, seborrhoeic dermatitis or skin disorders associated with hyperseborrhoea.

### Modulators of the Protein

A subject of the invention is also the use of a modulator of the human ADFP protein, that can be obtained by means of one of the methods above, for the preparation of a medicament for use in the preventive and/or curative treatment of acne, of seborrhoeic dermatitis or of skin disorders associated with hyperseborrhoea.

A method for the preventive and/or curative treatment of acne, of seborrhoeic dermatitis or of skin disorders associated with hyperseborrhoea is thus described herein, said method comprising the administration of a therapeutically effective amount of a modulator of the adipose differentiation-related protein to a patient requiring such a treatment.

Finally, the invention is directed towards the cosmetic use of a modulator of the adipose differentiation-related protein, for the aesthetic treatment of greasy skin.

Preferably, the modulator is an ADFP inhibitor. The term “inhibitor” refers to a compound or a chemical substance which eliminates or substantially reduces the biological activity of the adipose differentiation-related protein. The term “substantially” signifies a reduction of at least 25%, preferably of at least 35%, more preferably of at least 50%, and more preferably of at least 70% or 90%.

A preferred inhibitor interacts with ADFP in solution at inhibitor concentrations of less than 20 μM, less than 10 μM, less than 5 μM, less than 1 μM, preferably less than 0.1 μM, more preferably less than 0.01 μM.

The modulator compound may be an anti-ADFP inhibitory antibody, preferably a monoclonal antibody. Advantageously, such an inhibitory antibody is administered in an amount sufficient to obtain a plasma concentration of approximately 0.01 μg per ml to approximately 100 μg/ml, preferably of approximately 1 μg per ml to approximately 5 μg/ml.

The modulator compound may also be a polypeptide, an antisense DNA or RNA polynucleotide, an siRNA or a PNA (peptide nucleic acid, polypeptide chain substituted with purine and pyrimidine bases, the spatial structure of which mimics that of the DNA and enables hybridization thereto).

The modulator compound may also be an aptamer. The aptamer is a class of molecules representing, in terms of molecular recognition, an alternative to antibodies. They are oligonucleotide sequences which have the ability to recognize virtually all the classes of target molecules with a high affinity and specificity. Such ligands can be isolated by systematic evolution of ligand by exponential enrichment (SELEX) carried out on a library of random sequences, as described by Tuerk and Gold, 1990. The library of random sequences can be obtained by combinatorial chemical synthesis of DNA. In this library, each member is a linear, optionally chemically modified, oligomer of a unique sequence. Possible modifications, uses and advantages of this class of molecules have been reviewed in Jayasena, 1999, Clinical Chemistry 45(9): 1628-1650.

The invention comprises the use of such compounds that inhibit the adipose differentiation-related protein for the preventive and/or curative treatment of acne, of seborrhoeic dermatitis or of skin disorders associated with hyperseborrhoea. In a nonlimiting manner, an anti-ADFP antibody may be mentioned as inhibitor of the human ADFP protein.

Other modulator compounds identified by the screening method described above are also useful.

The modulator compounds are formulated within a pharmaceutical composition, in combination with a pharmaceutically acceptable carrier. These compositions may be administered, for example, orally, enterally, parenterally, or topically. Preferably, the pharmaceutical composition is applied topically. By oral administration, the pharmaceutical composition may be in the form of tablets, gel capsules, sugar-coated tablets, syrups, suspensions, solutions, powders, granules, emulsions, suspensions of microspheres or nanospheres or lipid or polymeric vesicles for controlled release. By parenteral administration, the pharmaceutical composition may be in the form of solutions or suspensions for a drip or for injection.

By topical administration, the pharmaceutical composition is more particularly for use in treating the skin and the mucous membranes and may be in the form of salves, creams, milks, ointments, powders, impregnated pads, solutions, gels, sprays, lotions or suspensions. It may also be in the form of suspensions of microspheres or nanospheres or lipid or polymeric vesicles or polymeric patches or hydrogels for controlled release. This composition for topical application may be in anhydrous form, in aqueous form or in the form of an emulsion. In a preferred variant, the pharmaceutical composition is in the form of a gel, a cream or a lotion.

The composition may comprise an ADFP-modulator content ranging from 0.001% to 10% by weight, in particular from 0.01% to 5% by weight, relative to the total weight of the composition.

The pharmaceutical composition may also contain inert additives or combinations of these additives, such as

wetting agents;

flavour enhancers;

preservatives such as para-hydroxybenzoic acid esters;

stabilizers;

moisture regulators;

pH regulators;

osmotic pressure modifiers;

emulsifiers;

UV-A and UV-B screens;

and antioxidants, such as alpha-tocopherol, butylhydroxyanisol or butylhydroxytoluene, superoxide dismutase, ubiquinol or certain metal chelating agents.

The following examples illustrate the invention without limiting the scope thereof.

### EXAMPLES

### Experimental Data

### Example 1

### Expression of the Adipose Differentiation-Related Protein in the Human Sebaceous Gland and in Human Epidermis

Human sebaceous glands were separated from human epidermis by treatment with dispase and dissection under a binocular magnifying lens. Total RNA samples were prepared from the sebaceous glands and from the epidermis.

The expression of the genes was analysed on an Affymetrix station (microfluidic module; hybridization oven; scanner; computer) according to the protocols supplied by the company. Briefly, the total RNA isolated from the tissues is transcribed into cDNA. A biotin-labelled cRNA is synthesized, from the double-stranded cDNA, using T7 polymerase and a precursor NTP conjugated to biotin. The cRNAs are subsequently fragmented into small fragments. All the molecular biology steps are verified using the Agilent “lab on a chip” system in order to confirm that the enzymatic reactions are very efficient. The Affymetrix chip is hybridized with the biotinylated cRNA, rinsed, and subsequently labelled by fluorescence using a Streptavidin-conjugated fluorophore. After washing, the chip is scanned and the results are calculated using the MAS5 software supplied by Affymetrix. An expression value is obtained for each gene, as is an indication of the significance of the value obtained. The calculation of the significance of the expression is based on the analysis of the signals which are obtained following hybridization of the cRNA of a given gene with a perfect match oligonucleotide versus an oligonucleotide which contains a single mismatch in the central region of the oligonucleotide (see Table 1).

TABLE 1
Measurement of the expression of the adipose
differentiation-related protein in the epidermis and in
the human sebaceous gland by the use of the Affymetrix
chip technology

| | | | | Significance | |
|---|---|---|---|---|---|
| | | Expression | | of the | Significance |
| | | in the | | expression* | of the |
| | | human | Expression | in the human | expression* |
| Affymetrix | Gene | sebaceous | in human | sebaceous | in human |
| identifier | name | gland | epidermis | gland | epidermis |
| 209122_at | adipose | 933 | 290 | 1 | 1 |
| | differen- | | | | |
| | tiation- | | | | |
| | related | | | | |
| | protein | | | | |

*indicator of the significance of the expression of the gene analysed in the sample indicated: presence (=1) or absence (=0).

### Example 2

### Expression of the Adipose Differentiation-Related Protein in the Human Sebaceous Gland and in Human Epidermis

The samples of epidermis and of human sebaceous gland were prepared by laser microdissection from three lifts of healthy human skin (female donors).

The expression of the messenger RNA encoding the ADFP protein was analysed by quantitative RT-PCR (qRT-PCR) using the microfluidics cards technology developed by Applied Biosystems.

The Ct corresponds to the number of PCR cycles which makes it possible to choose the same level of fluorescence for all the samples. The level of expression is represented in each tissue by the mean of the Cts and the standard deviation obtained on the three donors.

The differential expression between the two tissues is measured via a mean induction factor (I.F) for the sebaceous gland with respect to the epidermis after standardization of the Cts via the expression of the three housekeeping genes (ribosomal 18S RNA, glyceraldehyde 3-phosphate dehydrogenase GAPDH, beta-actin).

TABLE 2
qRT-PCR measurement of the expression of ADFP
in the epidermis and the human sebaceous gland via the
use of the microfluidic cards technology (Applied Biosystems)

| | Number of | | | | |
|---|---|---|---|---|---|
| | cycles | | Number of | | |
| | necessary | | cycles | | |
| | for | | necessary | | |
| | detecting | | for | | Mean induction |
| | the mean | | detecting | | factor for |
| | level of | | the mean | | expression in |
| | expression | | level of | | the sebaceous |
| | in the | | expression | | gland versus |
| | human | | in human | | human |
| Gene | sebaceous | Standard | epidermis | Standard | epidermis |
| name | gland (Ct) | deviation | (Ct) | deviation | (I.F) |
| adipose | 26 | 1.11 | 29 | 1.11 | 13 |
| differen- | | | | | |
| tiation- | | | | | |
| related | | | | | |
| protein | | | | | |

### Example 3

### Expression of the Adipose Differentiation-Related Protein in Human Sebocytes in Primary Culture

a. Isolation and Culture of Human Sebocytes

Human sebocytes are cultured using lifts from healthy human donors according to the method described by Xia et al. (J Invest Dermatol. 1989 September; 93(3):315-21) after separation of the epidermis from the dermis through the action of dispase and microdissection of the sebaceous glands under binocular magnifying lenses.

The sebaceous glands are seeded in 6-well plates on a feeder layer of mitomycin-treated 3T3 fibroblasts in DMEM-Ham's F12 (3:1) medium supplemented with 10% foetal calf serum (FCS); 10 ng/ml of epidermal growth factor (EGF); 10⁻¹⁰ M cholera toxin (CT); 0.5 μg/ml of hydrocortisone (HC); 5 μg/ml of insulin (INS); 2 mM L-glutamine (Gln); 100 IU/ml of penicillin-streptomycin (PS).

The first foci of human sebocytes appear 3 days after seeding of the glands.

The cells are then treated for 6 days with the sebogenic cocktail corresponding to the combination of PPARγ agonist rosiglitazone (1 μM) and the androgen R1881 (10 nM), or with dimethyl sulphoxide (DMSO) used as carrier.

b. PCR Expression Data

The expression of the messenger RNA encoding the ADFP protein was analysed by qRT-PCR using the microfluidics cards technology developed by Applied Biosystems, as described above (Example 2), on a culture of human sebocytes corresponding to one donor.

The level of expression (Ct) is represented for each treatment condition.

The induction of ADFP expression by the sebogenic cocktail is measured via an induction factor (I.F) versus the DMSO control after standardization of the Cts via the expression of the three housekeeping genes (ribosomal 18S RNA, glyceraldehyde 3-phosphate dehydrogenase GAPDH, beta-actin).

TABLE 3
qRT-PCR measurement of the expression of ADFP
in a primary culture of human sebocytes treated for 6
days with the sebogenic cocktail (combination of 1 μM
PPARγ agonist rosiglitazone; 10 nM androgen R1881) or
with DMSO, via the use of the microfluidic cards
technology (Applied Biosystems)

| | Number of | Number of | |
|---|---|---|---|
| | cycles | cycles | |
| | necessary for | necessary for | Induction factor |
| | detecting the | detecting the | for expression |
| | mean level of | mean level of | under the |
| | expression in | expression in | condition treated |
| | human | human sebocytes | with the sebogenic |
| | sebocytes | treated with | mix versus the |
| Gene | treated with | the sebogenic | DMSO condition |
| name | DMSO (Ct) | cocktail (Ct) | (I.F) |
| adipose | 31 | 27 | 11 |
| differen- | | | |
| tiation- | | | |
| related | | | |
| protein | | | |

### Example 4

### Expression of the Adipose Differentiation-Related Protein in the Rat Preputial Gland in Primary Culture

Primary cultures of rat preputial gland sebocytes (Rosenfield et al., J. Invest. Dermatol. 1999; 112:226-32) were used to evaluate differentiation cocktails such as the combination of PPARγ and an androgen receptor agonist. After seeding on 24-well plates, the preputial cells are cultured for 3 days in DMEM medium containing 10% of foetal calf serum (FCS), 10⁻¹⁰ M of cholera toxin (CT), 10⁻¹⁰ M of cortisol, 5 μg/ml of insulin and antibiotics. The cells are then cultured in a serum-free medium (Cellgro complete medium) and treated with the PPARγ agonist (rosiglitazone, 100 nM) and the androgen receptor agonist (R1181, 1 nM) for 3 to 9 days with the medium being changed every 2 days. The cells are recovered on the 9^{th} day and the large-scale analysis of the gene expression is carried out by means of Affymetrix RAE230A chips.

TABLE 4
Measurement of the expression of the adipose
differentiation-related protein in preputial gland
cells in culture in response to a cocktail of an
androgen (R1881 at 1 μM) and of a PPARγ ligand
(rosiglitazone at 100 nM) via the use of the Affymetrix
chip technology. The mixture is known to induce cell
differentiation characterized by increased lipogenesis

| | | Expres- | | Signifi- | Significance |
|---|---|---|---|---|---|
| | | sion | | cance | of the |
| | | under | Expression | of the | expression* |
| | | the | after | expres- | after |
| | | control | treatment | sion* | treatment |
| | | condi- | with R1881 | under the | with R1881 |
| Affymetrix | Gene | tion | and | control | and |
| identifier | name | (DMSO) | rosiglitazone | condition | rosiglitazone |
| 1390383_at | adipose | 1392 | 2849 | 1 | 1 |
| | differen- | | | | |
| | tiation- | | | | |
| | related | | | | |
| | protein | | | | |

*Indicator of the significance of the expression of the gene analysed in the sample indicated: presence (=1) or absence (=0).

1.-29. (canceled)

30. An in vitro or in vivo method for screening for candidate compounds for the preventive and/or curative treatment of acne, of seborrhoeic dermatitis or of skin disorders associated with hyperseborrhoea, comprises determining the ability of a compound to modulate the expression or the activity of the adipose differentiation-related protein (ADFP) or the expression of the gene thereof, or the activity of at least one of the promoters thereof.

31. An in vitro method for screening for candidate compounds for the preventive and/or curative treatment of acne, of seborrhoeic dermatitis or of skin disorders associated with hyperseborrhoea as defined by claim 30, comprising the following steps:
a. preparing at least two biological samples or reaction mixtures;
b. contacting one of the samples or reaction mixtures with one or more of the test compounds;
c. measuring the expression or the activity of the adipose differentiation-related protein, the expression of the gene thereof or the activity of at least one of the promoters thereof, in the biological samples or reaction mixtures; and
d. selecting the compounds for which a modulation of the expression or of the activity of the ADFP protein, or a modulation of the expression of the gene thereof or a modulation of the activity of at least one of the promoters thereof, is measured in the sample or the mixture treated in b) compared with the untreated sample or with the untreated mixture.

32. The in vitro method as defined by claim 31, wherein the compounds selected in step d) inhibit the expression or the activity of the adipose differentiation-related protein, the expression of the gene thereof or the activity of at least one of the promoters thereof.

33. The in vitro method as defined by claim 31, wherein the biological samples are cells transfected with a reporter gene functionally linked to all or part of the promoter of the gene encoding the adipose differentiation-related protein, and step c) comprises measuring the expression of said reporter gene.

34. The in vitro method as defined by claim 31, wherein the biological samples are cells expressing the gene encoding the adipose differentiation-related protein, and step c) comprises measuring the expression of said gene.

35. The in vitro method as defined by claim 33, wherein the cells comprise sebocytes.

36. The in vitro method as defined by claim 34, wherein the cells comprise cells transformed with a heterologous nucleic acid encoding the adipose differentiation-related protein.

37. The in vitro method as defined by claim 31, wherein the expression of the gene is determined by measuring the level of transcription of said gene.

38. The in vitro method as defined by claim 31, wherein the expression of the gene is determined by measuring the level of translation of said gene.

39. The in vitro or in vivo method as defined by claim 30, comprising contacting a compound with an ADFP protein and determining the ability of the compound to modulate ADFP, a difference in activity, compared to a control carried out in the absence of the compound, indicating the utility of the compound for the preventive or curative treatment of acne, of seborrhoeic dermatitis or of a skin disorder associated with hyperseborrhoea.

40. A medicament useful for the preventive and/or curative treatment of acne, of seborrhoeic dermatitis or of skin disorders associated with hyperseborrhoea, comprising a modulator of the human ADFP protein obtained by means of the method as defined by claim 31.

41. The medicament as defined by claim 40, wherein the modulator comprises an inhibitor of the ADFP protein.

42. The medicament as defined by claim 40, wherein the modulator comprises an siRNA.

43. A regime or regimen for the aesthetic treatment of greasy skin, comprising administering to an individual in need of such treatment, a thus effective amount of a modulator of the human ADFP protein.

44. An in vitro method for diagnosing or monitoring the development of acne, seborrhoeic dermatitis or a skin disorder associated with hyperseborrhoea in an individual, comprising comparing the expression or the activity of the adipose differentiation-related protein, the expression of the gene thereof or the activity of at least one promoter thereof, in a biological sample from an individual, with respect to a biological sample from a control individual.

45. The in vitro method as defined by claim 44, wherein the expression of the protein is determined by assaying this protein by immunoassay.

46. The in vitro method as defined by claim 45, wherein the immunoassay comprises an ELISA assay.

47. The in vitro method as defined by claim 44, wherein the expression of the gene is determined by measuring the amount of corresponding mRNA.

48. An in vitro method for determining an individual's susceptibility to developing acne, seborrhoeic dermatitis or a skin disorder associated with hyperseborrhoea, comprising comparing the expression or the activity of the ADFP protein, the expression of the gene thereof or the activity of at least one of the promoters thereof, in a biological sample from an individual, with respect to a biological sample from a control individual.

49. The in vitro method as defined by claim 48, wherein the expression of the protein is determined by assaying this protein by means of an immunoassay.

50. The in vitro method as defined by claim 49, wherein the immunoassay comprises an ELISA assay or a radioimmunoassay.

51. The in vitro method as defined by claim 48, wherein the expression of the gene is determined by measuring the amount of corresponding mRNA.

52. A marker for screening for candidate PPAR modulators for the treatment of acne, of seborrhoeic dermatitis or of a skin disorder associated with hyperseborrhoea, comprising the gene of the adipose differentiation-related protein or of the adipose differentiation-related protein.

53. The marker as defined by claim 52, for determining the ability of a PPAR modulator to modulate the expression or the activity of ADFP or the expression of the gene thereof or the activity of at least one of the promoters thereof.

54. The marker as defined by claim 52, wherein the PPAR modulator comprises a PPARγ modulator.

55. The marker as defined by claim 52, wherein the modulator comprises a PPAR receptor agonist.

56. A marker for screening for candidate AR (androgen receptor) modulators for the treatment of acne, of seborrhoeic dermatitis or of a skin disorder associated with hyperseborrhoea, comprising a gene encoding the adipose differentiation-related protein (ADFP) or of an adipose differentiation-related protein (ADFP).

57. The marker as defined by claim 56, for determining the ability of an AR modulator to modulate the expression or the activity of ADFP or the expression of the gene thereof or the activity of at least one of the promoters thereof.

58. The marker as defined by claim 56, wherein the modulator comprises an androgen receptor agonist.
